(19) United States
(12) Patent Application Publication
(10) Pub. No.: US 2025/0147025 A1
(43) Pub. Date: May 8, 2025

(54) METHODS AND KITS FOR DETERMINING THE PRESENCE AND/OR AMOUNT OF A HUMAN IGG3 ANTIBODY SPECIFIC FOR A FLAVIVIRUS ANTIGEN IN A SAMPLE

(71) Applicant: Takeda Vaccines, Inc., Cambridge, MA (US)

(72) Inventor: Eduardo Nascimento, Cambride, MA (US)

(21) Appl. No.: 18/722,290

(22) PCT Filed: Dec. 19, 2022
(86) PCT No.: PCT/US2022/081970

§ 371 Date: Jun. 20, 2024

(30) Foreign Application Priority Data
Dec. 21, 2021 (EP) 21216535.1

(51) Int. Cl. G01N 33/569

(52) U.S. Cl. CPC G01N 33/56983; G01N 2333/185; G01N 2469/20; G01N 2470/04

(57) ABSTRACT

The present invention relates to methods for determining the presence and/or amount of a human IgG3 antibody, preferably specific for a virus antigen in a sample. The present invention also relates to the use of the methods in the quality control of viral vaccines, and in the diagnosis of viral infections. Further, kits for use in the methods are described as well.

### CROSS-REFERENCE TO RELATED APPLICATIONS

This International PCT Application claims priority to and the benefit of European Application No. 21 216 535. 1 filed on 21 Dec. 2021, the contents of which is incorporated herein by reference in its entirety.

### TECHNICAL FIELD OF THE INVENTION

The present invention relates to methods for determining the presence and/or amount of a human IgG3 antibody, preferably specific for a virus antigen in a sample. The present invention also relates to the use of the methods in the quality control of viral vaccines, and in the diagnosis of viral infections. Further, kits for use in the methods are described as well.

### BACKGROUND OF THE INVENTION

Antibody responses to viral infections in humans are varied and of widely divergent clinical significance. Preexisting, reactive antibodies or antibodies that are formed early during infection can bind to virus particles, forming immune complexes that can neutralize or mediate clearance of the virus. On the other hand, immune complexes can also promote inflammation and exacerbate symptoms of disease. How antibodies within immune complexes modulate infection depends, in part, on their Fc domain structure. Fc structure, in turn, dictates interactions with Fcγ receptors (FcγRs), which are expressed by a variety of cells that are activated during infection.

Antibody isotypes IgG, IgA and IgM are a primary determinant of Fc structure and thus of activity. Initial B cell responses are characterized by the production of IgM antibodies. Production of class-switched IgA and IgG antibodies follows, with IgA playing a central role in mucosal immunity, while IgG is the dominant isotype involved in systemic antiviral immunity. IgG functions are governed by interactions between immune complexes and effector immune cells that express FcγRs, the receptors for IgG. The balance of FcγRs that are engaged by immune complexes determines the degree of the inflammatory effector cell response. Activating, low-affinity FcγRs (FcγRIIa and FcγRIIIa) transduce inflammatory signaling through immunoreceptor tyrosine-based activation motifs (ITAMs); in health, ITAM signaling is balanced by immunoreceptor tyrosine-based inhibition motif (ITIM) signaling through the inhibitory FcγRIIb.

The strength of interactions between immune complexes and various FcγRs is determined by structural diversity within IgG subclasses (IgG1, IgG2, IgG3 and IgG4) and posttranslational modifications of their Fc domains. Importantly, individuals produce distinct structural repertoires of IgG Fc domains, with some producing highly activating/proinflammatory repertoires enriched for features such as IgG1, IgG3 and/or reduced core fucosylation of the IgG1 Fc domain. Others produce IgG repertoires characterized by higher levels of IgG2 and/or sialylated Fcs that have reduced activating/inflammatory FcγR signaling potential (Chakaborty et al., Nature Immunol. 22 (2021), 67-73).

Nascimento et al., J. Virol. Methods 257 (2018), 62-68 describes that anti-dengue NS1-specific IgG and IgG3 are potential biomarkers of long-term and recent (less than 6 months) dengue virus infections, respectively. IgG3 was also used as a marker of recent infection by HIV (Viana et al., Epidemiology & Infection 146 (2018), 1293-1300. Rodriguez-Barraquer et al., Science 363 (6427), 607-610 describe the measurement of the IgG3 response against Zikavirus NS1 protein.

Trend et al., Front. Immunol. 9 (2018), Article 1590:1-13 outlines that higher serum IgG3 levels may predict the development of Multiple sclerosis in individuals with clinically isolated syndrome.

Chakaborty et al., supra, describes that IgG3 and IgG1 with F0N0 glycoform modification are elevated in more patients with severe COVID-19.

There is a need for the provision of an improved assay for determining IgG3 subtype antibodies. In particular, an assay shall be provided wherein the sensitivity and/or specificity is increased over prior art assays for determining IgG3, in particular human IgG3 specific for a virus antigen.

### SUMMARY OF THE INVENTION

In a first aspect, the present invention provides a method for determining the presence and/or amount of a human IgG3 antibody specific for a flavivirus antigen in a sample comprising the steps of:
Step 1: contacting an amount of the sample with Protein A coupled to beads to allow binding of human IgG1, human IgG2, human IgG4, human IgA, human IgE and human IgM antibodies to the Protein A coupled to the beads;
Step 2: separating the human IgG1, human IgG2, human IgG4, human IgA, human IgE and human IgM antibodies bound to the Protein A coupled to the beads from the remaining sample and thereby producing a human IgG3 antibody enriched supernatant;
Step 3: removing the human IgG3 antibody enriched supernatant from the human IgG1, human IgG2, human IgG4, human IgA, human IgE and human IgM antibodies bound to the Protein A coupled to the beads:
Step 4: contacting a virus antigen with the human IgG3 antibody enriched supernatant to allow binding of the human IgG3 antibody to the flavivirus antigen:
Step 5: contacting the human IgG3 antibody bound to the virus antigen with a primary IgG specific antibody or a primary IgG3 specific antibody to allow binding of the primary IgG specific antibody or the primary IgG3 specific antibody to the human IgG3 antibody bound to the flavivirus antigen:
Step 6: contacting the bound primary IgG3 specific antibody or the bound primary IgG specific antibody of step 4 with a labelled secondary antibody being specific for the primary IgG3 specific antibody or the primary IgG specific antibody to allow binding of the labelled secondary antibody to the primary antibody; and
Step 7: detecting a signal from the labelled secondary antibody bound to the primary antibody in step 5, wherein the signal is indicative for the presence and/or amount of the labelled secondary antibody and wherein the presence and/or amount of the labelled secondary antibody is indicative for the presence and/or amount of human IgG3 antibody specific for a flavivirus antigen in the sample.

In a second aspect the present invention provides the use of a pre-treatment of a sample containing human IgG3 specific for a flavivirus antigen and one or more of human IgG1, human IgG2, human IgG4, human IgA and human IgM antibodies with immobilized or immobilizable Protein A in vitro for increasing the sensitivity of a method for determining the presence and/or amount of the human IgG3 antibody in the sample.

In a third aspect, the present invention provides the use of the method according to the present invention for the in vitro diagnosis of a flavivirus infection within the last six months, preferably a dengue virus infection.

In a fourth aspect, the present invention provides the use of the method according to the present invention for determining the human IgG3 response to a flaviviral vaccine.

In a fifth aspect, the present invention provides a kit for determining the presence and/or amount of human IgG3 antibody in a sample comprising
(a) Protein A coupled to magnetic beads;
(b) a primary IgG specific antibody or a primary IgG3 specific antibody; and
(c) a labelled secondary antibody specific for the primary IgG specific antibody or the primary IgG3 specific antibody.

In a sixth aspect, the present invention provides a kit for determining the presence and/or amount of human IgG3 antibody in a sample comprising
(a) Protein A coupled to magnetic beads; and
(b) a labelled primary IgG specific antibody or a labelled primary IgG3 specific antibody.

The prior art methods for determining IgG3 subtype antibodies such as ELISAs or immunofluorescent assays are based on the use of an IgG3 specific primary antibody. The proportion of IgG3 in biological samples is relatively low, generally below 5%. In contrast, the proportion of IgG1 and IgG2 is 60% and 32%, respectively; see Vidarsson et al., Front. Immunol. 5 (2014), Article 520:1-17. The present inventors have found that due to the co-presence of high amounts of IgG1 and IgG2 the sensitivity/specificity of the prior art assays is negatively affected. The present inventors have found that by a prior step of removing of human IgM, human IgA, human IgG1, human IgG2 and human IgG4 by the use of Protein A the sensitivity of the detection of human IgG3 in a sample can be significantly increased.

### BRIEF DESCRIPTION OF THE DRAWINGS

FIG. 1 Determination of the IgG3 amount in different patient samples with the method according to the present invention (designated as Protein A) and a method which did not include the Protein A depletion step before the actual determination of IgG3 (designated as pre-depletion). DENV1 VLP (upper left panel). DENV2 VLP (upper right panel), DENV3 VLP (lower left panel) and DENV4 VLP (lower right panel), respectively, have been used as antigen. Presumed Dengue samples used in FIGS. 1 to 6: PLA-102, PLA-116, PLA-108, and PLA-117. Presumed Zika samples used in FIGS. 1 to 6: PARS_64, PARS_55, PARS_71, PARS_70, and PARS_97.

FIG. 2 Determination of the IgG3 amount in different patient samples with the method according to the present invention (designated as Protein A) and a method which did not include the Protein A depletion step before the actual determination of IgG3 (designated as pre-depletion). DENV1 NS1 protein (upper left panel), DENV2 NS1 protein (upper right panel). DENV3 NS1 protein (lower left panel), and DENV4 NS1 protein (lower right panel), respectively, have been used as antigen.

FIG. 3 Determination of the IgG3 amount in different patient samples with the method according to the present invention (designated as Protein A) and a method which did not include the Protein A depletion step before the actual determination of IgG3 (designated as pre-depletion). DENV1 VLP (upper left panel). DENV2 VLP (upper right panel), DENV3 VLP (lower left panel) and DENV4 VLP (lower right panel), respectively, have been used as antigen.

FIG. 4 Determination of the IgG3 amount in different patient samples with the method according to the present invention (designated as Protein A) and a method which did not include the Protein A depletion step before the actual determination of IgG3 (designated as pre-depletion). DENV1 NS1 protein (upper left panel), DENV2 NS1 protein (upper right panel). DENV3 NS1 protein (lower left panel), and DENV4 NS1 protein (lower right panel), respectively, have been used as antigen.

FIG. 5 Determination of the IgG3 amount in different patient samples with the method according to the present invention (designated as Protein A) and a method which did not include the Protein A depletion step before the actual determination of IgG3 (designated as pre-depletion). Zika VLP has been used as antigen.

FIG. 6 Determination of the IgG3 amount in different patient samples with the method according to the present invention (designated as Protein A) and a method which did not include the Protein A depletion step before the actual determination of IgG3 (designated as pre-depletion). Zika NS1 protein has been used as antigen.

### DETAILED DESCRIPTION OF THE INVENTION

In a first aspect the present invention provides a method for determining the presence and/or amount of a human IgG3 antibody specific for a flavivirus antigen in a sample comprising the steps of:
Step 1: contacting an amount of the sample with Protein A coupled to beads to allow binding of human IgG1, human IgG2, human IgG4, human IgA, human IgE and human IgM antibodies to the Protein A coupled to the beads:
Step 2: separating the human IgG1, human IgG2, human IgG4, human IgA, human IgE and human IgM antibodies bound to the Protein A coupled to the beads from the remaining sample and thereby producing a human IgG3 antibody enriched supernatant:
Step 3: removing the human IgG3 antibody enriched supernatant from the human IgG1, human IgG2, human IgG4, human IgA, human IgE and human IgM antibodies bound to the Protein A coupled to the beads:
Step 4: contacting a virus antigen with the human IgG3 antibody enriched supernatant to allow binding of the human IgG3 antibody to the flavivirus antigen:
Step 5: contacting the human IgG3 antibody bound to the virus antigen with a primary IgG specific antibody or a primary IgG3 specific antibody to allow binding of the primary IgG specific antibody or the primary IgG3 specific antibody to the human IgG3 antibody bound to the flavivirus antigen;
Step 6: contacting the bound primary IgG3 specific antibody or the bound primary IgG specific antibody of step 4 with a labelled secondary antibody being specific for the primary IgG3 specific antibody or the primary IgG specific antibody to allow binding of the labelled secondary antibody to the primary antibody; and
Step 7: detecting a signal from the labelled secondary antibody bound to the primary antibody in step 5, wherein the signal is indicative for the presence and/or amount of the labelled secondary antibody and wherein the presence and/or amount of the labelled secondary antibody is indicative for the presence and/or amount of human IgG3 antibody specific for a flavivirus antigen in the sample.

Step 1 of the method according to the invention comprises: contacting an amount of the sample with Protein A coupled to beads to allow binding of human IgG1, human IgG2, human IgG4, human IgA, human IgE and human IgM antibodies to the Protein A coupled to the beads.

“Protein A” herein includes Protein A as such. Protein A is a 49 kDa surface protein originally found in the cell wall of the bacterium Staphylococcus aureus. It is encoded by the spa gene and its regulation is controlled by DNA topology, cellular osmolarity, and a two-component system called ArlS-ArlR. It has found use in biochemical research because of its ability to bind immunoglobulins. The term “protein A” herein also includes sequence variants of Protein A, which differ by the addition, substitution and/or deletion of at least one amino acid, preferably not more than 10 amino acids, more preferably not more than 5 amino acids and most preferably by not more than one amino acids. The term also includes conjugates of Protein A with peptides or proteins.

Protein A coupled to beads can be obtained commercially. Alternatively, it can be prepared by coupling of Protein A to beads. The beads are commercially available. The material of the beads may be sepharose or sephadex. The coupling may be effected by using activated moieties on the surface of the beads. Alternatively, magnetic beads may be used. Protein A coupled to magnetic beads is commercially available (AmMag™ Protein A Magnetic beads; Genscript).

“Sample” herein includes blood samples. Preferably, the sample is serum from a human individual. The individual may be virus infected patient or a healthy individual immunized with a viral vaccine. The virus may be a DNA or RNA virus. The virus may be either double-stranded or single-stranded.

More preferably, the DNA virus is a herpesvirus, a pox virus, an adenovirus, a papillomavirus and a polyomavirus.

More preferably, the RNA virus is a flavivirus, a reovirus, a retrovirus, an orthomyxovirus, a deltavirus, a rhabodovirus, a filovirus or a paramyxovirus. Even more preferably, the RNA virus is dengue virus, Yellow fever virus, Japanese encephalitis virus, Tick-Borne encephalitis virus, West Nile virus, Zika virus. HIV virus, influenza virus, rotavirus, RSV, coronavirus, measles, mumps or rabies. Most preferred the RNA virus is dengue virus, Yellow fever virus, West Nile virus, Zika virus and coronavirus. In particular, the RNA virus is dengue virus or a Zika virus. If the RNA virus is dengue virus, the virus may be selected from DENV1, DENV2, DENV3 and/or DENV4.

The vaccine used for vaccination of the individual may preferably be a dengue vaccine, e.g. a tetravalent vaccine such as TAK-003 (Takeda).

The contacting step can be performed in plate format or in tube format. Alternatively, it could be performed in a column format. Preferably, plates with 24 or 96 wells are used. In a first step the plates are incubated with the viral antigen to allow binding of human antibodies other than human IgG3 to the Protein A-coupled beads.

Conditions for binding of the human IgG1, human IgG2, human IgG4, human IgA, human IgE and human IgM to Protein A coupled to the beads are known to the skilled person. Strong acidic or basic conditions should be avoided in order to prevent denaturation of Protein A and/or the human antibodies. Preferably, the protein A loading takes place at a pH in the range from about pH 8.0 to about pH 10.0. More preferably, the loading takes place at pH 9.0 in 1 M potassium phosphate.

Step 2 of the method according to the invention comprises: separating the human IgG1, human IgG2, human IgG4, human IgA, human IgE and human IgM antibodies bound to the Protein A coupled to the beads from the remaining sample and thereby producing a human IgG3 antibody enriched supernatant.

The separation of the antibody bound Protein A being coupled to the beads from the remaining sample can be effected by centrifugation. If the beads are magnetic beads, the separation may be effected by magnetic force. This can be accomplished e.g. by using a magnetic stand.

Step 3 of the method according to the invention comprises: removing the human IgG3 antibody enriched supernatant from the human IgG1, human IgG2, human IgG4, human IgA, human IgE and human IgM antibodies bound to the Protein A coupled to the beads.

The remaining sample depleted of human IgG1, human IgG2, human IgG4, human IgA, human IgE and human IgM is then removed from the beads. This may be effected by aspiration or decanting.

Step 4 of the method according to the invention comprises: contacting a flavivirus antigen with the human IgG3 antibody enriched supernatant to allow binding of the human IgG3 antibody to the virus antigen.

A “virus antigen” herein refers to any substance which can be bound by an Ab. Antigens may induce an immune response within a subject. An antigen may have one or more epitopes. An antigen may be a protein, polypeptide, carbohydrate, polynucleotide, lipid, or combinations thereof. An antigen may be a truncated version of a protein, a protein tagged with an affinity tag such as a His- or a STREP-tag, or a single domain of a protein. As used herein, antigen may e.g. refer to a DENV1 VLP, DENV2 VLP, DENV3 VLP, DENV4 VLP, ZIKV VLP: ZIKV NS1. DENV1 NS1, DENV2 NS1, DENV3 NS1, DENV4 NS1, DENV1 E, DENV2 E, DENV3 E and/or DENV4 E. Consequently, the term flavivirus antigen refers to an antigen from a flavivirus.

The terms “virus like particle (VLP)” or “virus like particles (VLPs)” refer to molecules that closely resemble viruses, but are non-infectious because they do not contain viral genetic material. VLPs can be prepared recombinant through the expression of viral structural proteins, which can then self-assemble into the VLPs. Examples of VLPs are ZIKV VLPs and DENV VLPs.

The contacting step can be performed in plate format. Preferably, 48 well or 96 well plates may be used. In a first step the plates are incubated with the viral antigen to allow binding of the viral antigen to the surface of the plate. If different viral antigens e.g. from different flaviviruses such as dengue or Zika. or antigens from different dengue virus serotypes are to be tested, the antigens may be in different wells (singleplex format) or in the same well (multiplex format). Methods for carrying out singleplex and multiplex assays are known to the skilled person.

The contacting step may be followed by a blocking step known to the person skilled in the art. The conditions for incubation and blocking must be such that the viral antigen does not significantly denature. Any of these steps may be followed by a washing step.

Step 5 of the method according to the invention comprises: contacting the human IgG3 antibody bound to the flavivirus antigen with a primary IgG specific antibody or a primary IgG3 specific antibody to allow binding of the primary IgG specific antibody or the primary IgG3 specific antibody to the human IgG3 antibody bound to the flavivirus antigen.

The primary IgG specific antibody is an anti-IgG antibody capable of specifically binding to more than one IgG subtype. Preferably, the primary IgG specific antibody is a pan-IgG antibody specifically binding to each of the IgG subtypes, i.e. IgG1, IgG2, IgG3 and IgG4. Pan-IgG antibodies are commercially available.

Alternatively, a primary IgG3 specific antibody may be used. Such antibodies are commercially available as well or can be produced in a manner known in the art. Preferably, the primary IgG specific antibody and the primary IgG3 specific antibody is of non-human origin. More preferably, a mouse, rat, hamster of goat antibody may be used.

In a separate embodiment the primary IgG specific antibody or the primary IgG3 specific antibody is coupled to a detectable label. Accordingly, in this embodiment Steps 5 to 7 as defined above are replaced by

Step 5′: contacting the human IgG3 antibody bound to the virus antigen with a labelled primary IgG specific antibody or a labelled primary IgG3 specific antibody to allow binding of the labelled primary IgG specific antibody or the labelled primary IgG3 specific antibody to the human IgG3 antibody bound to the flavivirus antigen; and

Step 6′: detecting a signal from the labelled primary IgG specific antibody or a labelled primary IgG3 specific antibody bound to the virus antigen in step 5′, wherein the signal is indicative for the presence and/or amount of the labelled primary IgG specific antibody or a labelled primary IgG3 specific antibody and wherein the presence and/or amount of the labelled primary antibody is indicative for the presence and/or amount of human IgG3 antibody specific for a virus antigen in the sample.

Step 6 of the method according to the invention comprises: contacting the bound primary IgG3 specific antibody or the bound primary IgG specific antibody of step 4 with a labelled secondary antibody being specific for the primary IgG3 specific antibody or the primary IgG specific antibody to allow binding of the labelled secondary antibody to the primary antibody.

If the primary IgG specific antibody or the primary IgG3 specific antibody is not labelled, the secondary antibody being specific for the primary IgG specific antibody or the primary IgG3 specific antibody is labelled.

The term “label”, as used herein, refers to any compound or moiety that comprises one or more appropriate chemical substances or enzymes, which directly or indirectly generate a detectable compound or signal in a chemical, physical or enzymatic reaction. Labeling can be achieved by methods well known in the art (see, for example, Lottspeich, F., and Zorbas H., Springer Spektrum 2012, Bioanalytik).

As used herein, the term “labelled antibody”, refers to an Ab that is connected to a detectable label. The connection can be a covalent connection, which occurs for instance upon formation of an amide bound between the antibody and the detectable label. The type of connection is dependent on the functional groups available on the Ab and the label. In preferred embodiments, the antibody is attached to the label with the heavy chain constant region of the antibody.

The label may be selected from a chemiluminescent, fluorescent or an enzyme label. The enzyme may be horseradish peroxidase, alkaline oxidase, glucose oxidase or alkaline phosphatase. The substrate for the alkaline phosphatase may be adamantyl 1 or 2-dioxetane aryl phosphate (AMPPD) and for horseradish peroxidase the substrate may be luminol or its derivatives as a substrate.

In a preferred embodiment the label may be a fluorescence label selected from the group consisting of xanthene, fluorescein isothiocyanate, rhodamine, phycoerythrin, cyanine, coumarin, and any derivative thereof.

In a further preferred embodiment the label may be a chemiluminescent label selected from acridinium and ruthenium esters.

Step 7 of the method according to the invention comprises: detecting a signal from the labelled secondary antibody bound to the primary antibody in step 5, wherein the signal is indicative for the presence and/or amount of the labelled secondary antibody and wherein the presence and/or amount of the labelled secondary antibody is indicative for the presence and/or amount of human IgG3 antibody specific for the flavirus antigen in the sample.

The signal from the labelled secondary antibody is detected using a detection system. Any system which is suitable for determining values indicative for the presence and/or amount of the labelled secondary antibody bound to the bound primary IgG specific or primary IgG3 specific antibody may be used. The detection system comprises one or more light sources. Detection systems are commercially available and well known to the skilled person.

In a further aspect the present invention provides the use of a pre-treatment of a sample containing human IgG3 and one or more of human IgG1, human IgG2, human IgG4, human IgA and human IgM antibodies with immobilized or immobilizable Protein A in vitro for increasing the sensitivity of a method for determining the presence and/or amount of the human IgG3 antibody specific for a flavivirus antigen in the sample. The pre-treatment step may be carried out as described above for steps 1 to 3. Protein A may be immobilized by a covalent or a non-covalent bond to a solid support. Preferably, Protein A may be coupled to beads as outlined above. The sample may be a blood sample from a human subject. Preferably, the sample may contain human serum. The sample may further contain water, buffer salts and/or protein stabilizers.

In a further aspect the present invention provides the use of immobilized or immobilizable Protein A for the enrichment of human IgG3 in a biological sample containing human IgG3 and one or more of human IgG1, human IgG2, human IgG4, human IgA and human IgM antibodies.

In a further aspect the present invention provides the use of the method according to the invention for determining the human IgG3 response to a viral vaccine. The vaccine used for vaccination of the individual may preferably be a dengue or Zika vaccine, e.g. a tetravalent dengue vaccine such as TAK-003 (Takeda). In a preferred embodiment the human IgG3 response to at least one, preferably each of the different dengue serotypes is determined. The dengue serotypes include DENV1, DENV2, DENV3 and DENV4.

In a further aspect the present invention provides the use of the method according to the invention for the in vitro diagnosis of a flavivirus infection in a human subject within the last six months, preferably a dengue virus or Zika virus infection. In a preferred embodiment the human IgG3 response to at least one, preferably, each of the different dengue serotypes is determined. The dengue serotypes include DENV1, DENV2. ′DENV3 and DENV4.

Dengue virus infections and Zika virus infections result mostly in asymptomatic symptoms and as such can be difficult, without laboratory confirmation, making accurate estimates of the disease incidence. Direct confirmation of infection using tools such as PCR and detection of NS1 antigen have limited time windows (within the first few days after the onset of symptoms) for maximum sensitivity. In contrast, anti-dengue NS1-specific IgG3 is detected very early after the onset of symptoms and has a wider detection window (4 to 6 months).

In a separate preferred embodiment the human subject is infected by at least two different flaviviruses. Preferably, the human subject is infected by a dengue virus and a Zika virus. The infection may be acute or convalescent. For instance, the subject has been infected by DENV first and was then infected by ZIKV several months later. The in vitro method for diagnosing of the present application is capable of diagnosing the at least two different flavivirus infections e.g. the DENV and the ZIKV infection. Consequently, the in vitro method for diagnosing of the present application is capable of determining whether a subject was infected with one or more flaviviruses and by which flaviviruses the subject was infected. This can be very useful in practice as multiple infections with different flaviviruses are commonly observed as flaviviruses are co-circulating in several areas.

Alternatively, the human subject is infected by at least two different dengue virus serotypes. For instance, the subject can be infected by DENV serotype 1 and DENV serotype 2. The infection may be acute or convalescent. For instance, the subject has been infected by DENV serotype 1 first and was then infected by DENV serotype 2 several months later. The in vitro method for diagnosing of the present application is capable of diagnosing the at least two different dengue virus serotype infections e.g. the DENV serotype 1 and the DENV serotype 2 infection. Consequently, the in vitro method for diagnosing of the present application is capable of determining whether a subject was infected with one or more DENV serotypes and by which DENV serotypes the subject was infected.

In a further aspect the present invention provides kits for determining the presence and/or amount of human IgG3 antibody in a sample.

In one embodiment the kit for determining the presence and/or amount of human IgG3 antibody in a sample comprises:
(a) Protein A coupled to magnetic beads;
(b) a primary IgG specific antibody or a primary IgG3 specific antibody; and
(c) a labelled secondary antibody specific for the primary IgG specific antibody or the primary IgG3 specific antibody.

In a further embodiment the kit for determining the presence and/or amount of human IgG3 antibody in a sample comprises:
(a) Protein A coupled to magnetic beads;
(b) a labelled primary IgG specific antibody or a labelled primary IgG3 specific antibody

Specific embodiments for beads, IgG specific antibodies and labels are as defined above. The kit may include further components. It may also include instructions for the user.

### ITEMS OF THE INVENTION

1. A method for determining the presence and/or amount of a human IgG3 antibody specific for a virus antigen in a sample comprising the steps of:
   Step 1: contacting an amount of the sample with Protein A coupled to beads to allow binding of human IgG1, human IgG2, human IgG4, human IgA, human IgE and human IgM antibodies to the Protein A coupled to the beads;
   Step 2: separating the human IgG1, human IgG2, human IgG4, human IgA, human IgE and human IgM antibodies bound to the Protein A coupled to the beads from the remaining sample and thereby producing a human IgG3 antibody enriched supernatant;
   Step 3: removing the human IgG3 antibody enriched supernatant from the human IgG1, human IgG2, human IgG4, human IgA, human IgE and human IgM antibodies bound to the Protein A coupled to the beads:
   Step 4: contacting a virus antigen with the human IgG3 antibody enriched supernatant to allow binding of the human IgG3 antibody to the virus antigen;
   Step 5: contacting the human IgG3 antibody bound to the virus antigen with a primary IgG specific antibody or a primary IgG3 specific antibody to allow binding of the primary IgG specific antibody or the primary IgG3 specific antibody to the human IgG3 antibody bound to the virus antigen;
   Step 6: contacting the bound primary IgG3 specific antibody or the bound primary IgG specific antibody of step 4 with a labelled secondary antibody being specific for the primary IgG3 specific antibody or the primary IgG specific antibody to allow binding of the labelled secondary antibody to the primary antibody; and
   Step 7: detecting a signal from the labelled secondary antibody bound to the primary antibody in step 5, wherein the signal is indicative for the presence and/or amount of the labelled secondary antibody and wherein the presence and/or amount of the labelled secondary antibody is indicative for the presence and/or amount of human IgG3 antibody specific for a virus antigen in the sample.
2. The method of item 1, wherein the IgG3 antibody is specific for a flavivirus antigen, more preferably for a dengue virus antigen.
3. The method according to item 1 or 2, wherein the antigen is selected from the group consisting of virus like particle (VLP), non-structural protein 1, envelope protein, pre-membrane protein, membrane protein, capsid protein, non-structural protein 2A, non-structural protein 2B, non-structural protein 3, non-structural protein 4A, non-structural protein 4B, and non-structural protein 5 and any derivative thereof, preferably the antigen is VLP.
4. The method according to any one of items 1 to 3, wherein step 1 is carried out in microplate format, preferably in 96 well microplates.
5. The method according to any one of items 1 to 4, wherein the protein A is coupled to magnetic beads and the separating step 2 is performed by applying magnetic force.
6. The method according to any one of items 1 to 5, wherein the protein A is coupled to beads and the separating step 2 is performed by gravitational force, preferably by applying a step of centrifugation.
7. The method according to any one of items 1 to 6, wherein the human IgG3 antibody is enriched in the IgG3 supernatant compared to the content in the starting sample by a factor of at least 2, preferably at least 5, more preferably at least 10.
8. The method according to any one of items 1 to 7, wherein the label of the secondary antibody in step 6 includes a chemiluminescent, fluorescent or an enzyme label.
9. The method according to item 8, wherein the enzyme is horseradish peroxidase, alkaline oxidase or glucose oxidase.
10. The method according to item 8, wherein the label is a fluorescence label selected from the group consisting of xanthene, fluorescein isothiocyanate, rhodamine, phycoerythrin, cyanine, coumarin, and any derivative thereof.
11. The method according to any one of items 1 to 10, wherein steps 4 to 6 are performed in the format of a sandwich assay.
12. The method according to any one of items 1 to 11, wherein steps 4 to 7 are performed in microplate format, preferably in the format of 96 well plates.
13. Use of the method according to any one of items 1 to 12 for determining the human IgG3 response to a viral vaccine.
14. Use of the method according to any one of items 1 to 12 for the in vitro diagnosis of a virus infection within the last six months, preferably a dengue virus infection.
15. A kit for determining the presence and/or amount of human IgG3 antibody in a sample comprising
   (a) Protein A coupled to beads;
   (b) a primary IgG specific antibody or a primary IgG3 specific antibody; and
   (c) a labelled secondary antibody specific for the primary IgG specific antibody or the primary IgG3 specific antibody.
16. The kit according to item 15, wherein the protein A is coupled to magnetic beads.
17. The kit according to item 15 or 16, wherein the label of the secondary antibody in step 5 includes a chemiluminescent, fluorescent or an enzyme label.
18. The kit according to item 17, wherein the enzyme is horseradish peroxidase, alkaline oxidase or glucose oxidase.
19. The kit according to item 17, wherein the label is a fluorescent label selected from the group consisting of xanthene, fluorescein isothiocyanate, rhodamine, phycoerythrin, cyanine, coumarin, and any derivative thereof.

### EXAMPLES

### Materials and Methods

Orbital Plate Shaker Heidolph set was used. Protein A beads-AmMag™ Protein A Magnetic Beads were commercially obtained (Genscript; Cat. No. L00695). The magnetic plate was obtained from Life Technology (Cat. No. 032513). The Benchtop centrifuge was obtained from Thermo Scientific (Tag #305395: S/N 42204931). The microplates were obtained from Costar (Ref. No. 39155). The Dynamag magnetic stand for 2 mL and 50 mL tubes was obtained from Invitrogen (Cat. Nos. 12321D and 12302D, respectively).

### Samples

As samples, Presumed samples have been obtained commercially. Dengue samples: PLA-102, PLA-116, PLA-108, PLA-117; and as Zika samples: PARS_64. PARS_55, PARS_71, PARS_70, PARS_97. Samples were purchased from the company ABO Pharmaceuticals.

### Preparation of the Magnetic Beads

Before use the magnetic beads were resuspended and the resulting slurry transferred to a 50 ml conical tube. The magnetic beads were washed twice using a binding/wash buffer DPBS (1×) (Gibco). Then, the magnetic beads were washed twice with 0.1 N NaOH. Thereafter, the washing in binding/wash buffer as described above, was repeated twice.

Depletion of Human Antibodies Other than Human IgG3 from the Samples

The bead suspension pre-treated as described above was resuspended. 120 μl of the suspension are provided to each of the wells of a 96-well plate according to a pre-determined plate layout. The plate was put on a magnetic plate for at least 30 seconds and then flicked to remove the supernatant. 70 μl of sample were applied to the wells of the microplate according to the pre-determined plate layout and mixed with a pipet. The plates were incubated with mixing on a plate shaker (600 rpm) overnight (16-20 hours) at 37° C. The plates were spun down at 1000 rpm for one minute at 4° C. The suspension was transferred to 0.5 ml tube and a magnetic stand was used to separate the beads from the samples. The supernatant was carefully removed from the suspension in the plates. The supernatant was then further analyzed for the presence of IgG3 as described below.

### Detection of Anti-Dengue NS1-Specific IgG3 Antibodies and Anti-Zika NS1 Specific IgG3 Antibodies by ELISA

Half area 96-well plates (Corning. USA) were coated ovemnight, at 4″C, with either ZIKV NS1 (for ZIKV NS1 IgG3 assay, at equimolar ratio of all DENV scrotypes (for DENV NS 1 IgG3 assay: Native Antigen, UK) at 2.27 ug/mL. Plates were then blocked for 15 minutes at room temperature (RT). Serum samples and assay controls were diluted at 1:50 in blocking/dilution buffer, added to the wells in duplicate, and allowed to incubate for 1 hr at RT. Sera from a recent ZIKV infection, which were collected 20-30 days post onset of symptoms, was used as ZIKV positive control. Pooled sera from recent dengue infection, which were collected 20-30 days post onset of symptoms, was used as DENV positive control. Pooled sera from healthy individuals naïve to both ZIKV and DENV was used as negative control on both ZIKV and DENV assays. After washing 5 times, horseradish peroxidase (HRP)-conjugated mause monoclonal antibody anti human IgG3 (Invitrogen, USA) was added to wells and incubated for Ihr at RT. After another washing cycle, TMB (KPL, USA) was added to the wells and allowed to incubate for 30 minutes at RT. Reaction was stopped with 1N HCl (Sigma, USA) and optical densities were determined at wavelength of 450 nm (OD450) using SpectraMax Plus PC380 microplate spectrophotometer and SoftMax Pro software version 6.4 (Molecular Devices, USA). OD450 of sample and control wells were subtracted from the average of OD450 of the conjugate blank wells before analysis.

For DENV IgG3 analysis, DENV ratio was determined by dividing the OD450 of the samples by the average OD450 nm of the negative control. For ZIKV IgG3 analysis. ZIKV ratio was determined by dividing the OD450 of the samples by the average OD450 of the DENV recent infection.

The cut-off values for the DENV IgG3 and ZIKV IgG3 ratios were 1.5 and 0.586, respectively. A sample was defined as eligible for analysis ifthe average ratio of a sample was above the cut-off value (antigen-specific IgG3 positive sample) and coefficient ofvariation [(Standard deviation of replicates/average of replicates)×100] of replicates was below 20%.

### Detection of Anti-Dengue VLP-Specific IgG3 Antibodies and Anti-Zika VLP Specific IgG3 Antibodies by ELISA

The same procedure as outlined above was followed with the difference that Dengue VLP and Zika VLP, respectively, as antigen, instead of the NS1 antigen described above.

### Results

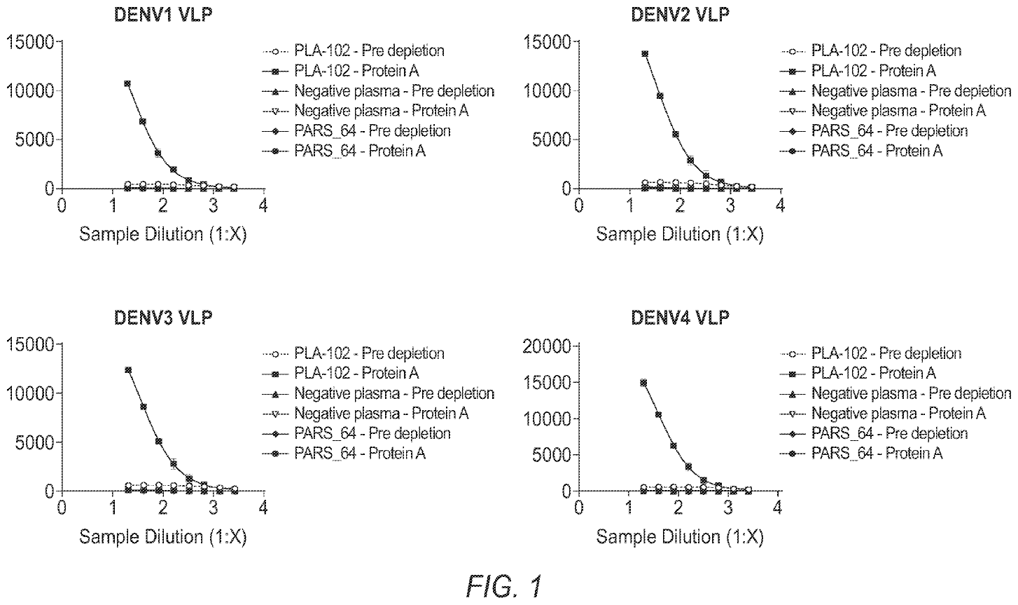

FIG. 1 shows a comparison of the results which have been obtained with the method according to the present invention (designated as Protein A) with a method which did not include the Protein A depletion step before the actual determination of IgG3 (designated as pre-depletion). It can be seen that for each of the four different DENV VLPs as antigen the method according to the invention resulted in a significantly increased sensitivity compared to the method without the prior Protein A depletion step.

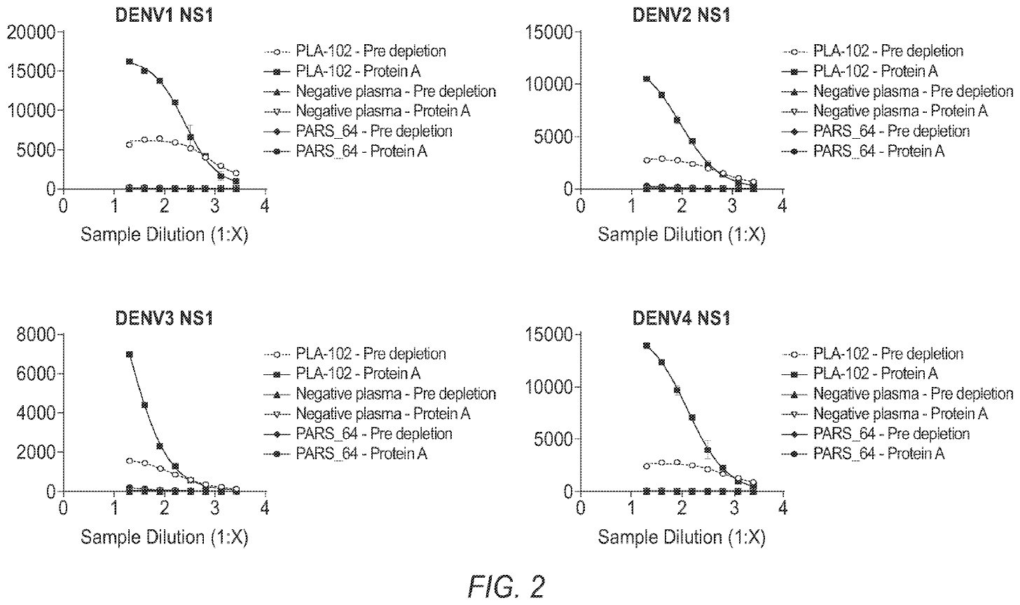

FIG. 2 shows a comparison of the results which have been obtained with the method according to the present invention (designated as Protein A) with a method which did not include the Protein A depletion step before the actual determination of IgG3 (designated as pre-depletion). It can be seen that for each of the four different DENV NS1 proteins as antigen the method according to the invention resulted in a significantly increased sensitivity compared to the method without the prior Protein A depletion step.

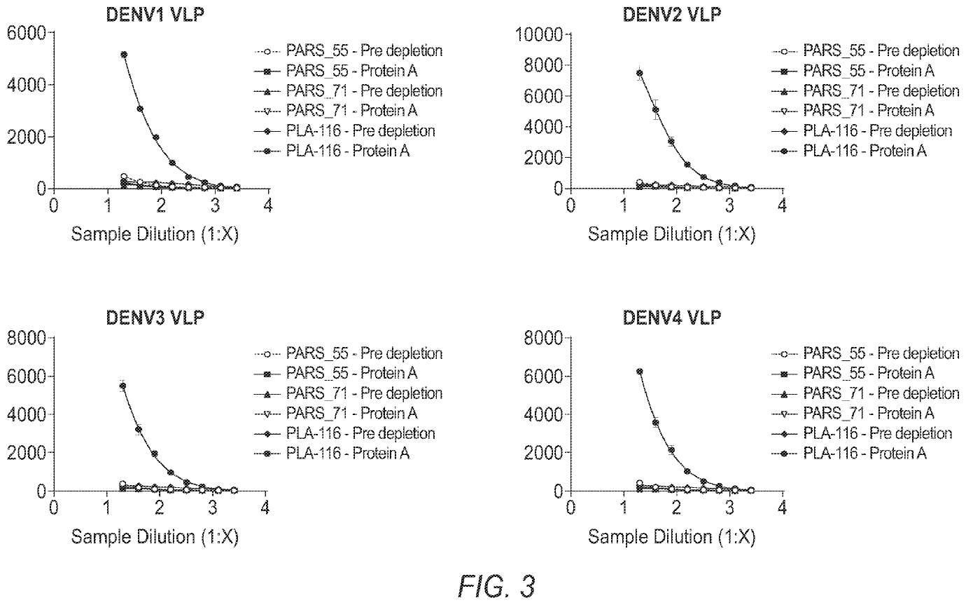

FIG. 3 relates to a comparison of the results which have been obtained with the method according to the present invention (designated as Protein A) with a method which did not include the Protein A depletion step before the actual determination of IgG3 (designated as pre-depletion). FIG. 3 differs from FIG. 1 in that different patient samples have been tested, but the same four different DENV VLPs have been used as antigens. Also here, the method according to the invention resulted in a significantly increased sensitivity.

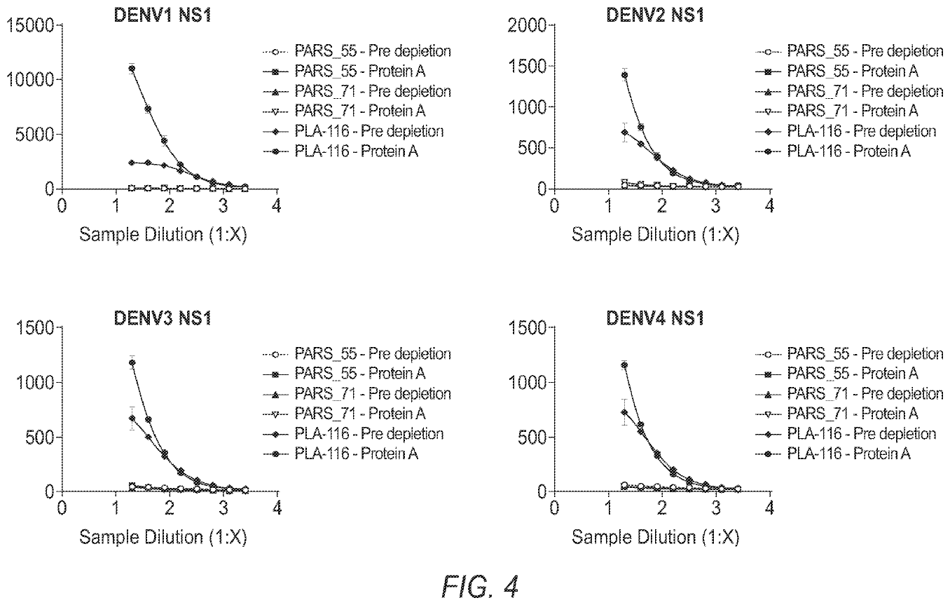

FIG. 4 shows a comparison of the results which have been obtained with the method according to the present invention (designated as Protein A) with a method which did not include the Protein A depletion step before the actual determination of IgG3 (designated as pre-depletion). FIG. 4 differs from FIG. 1 in that different patient samples have been tested, but the same four different DENV NS1 proteins as antigen have been used as antigens. The method according to the invention also provided a significantly increased sensitivity.

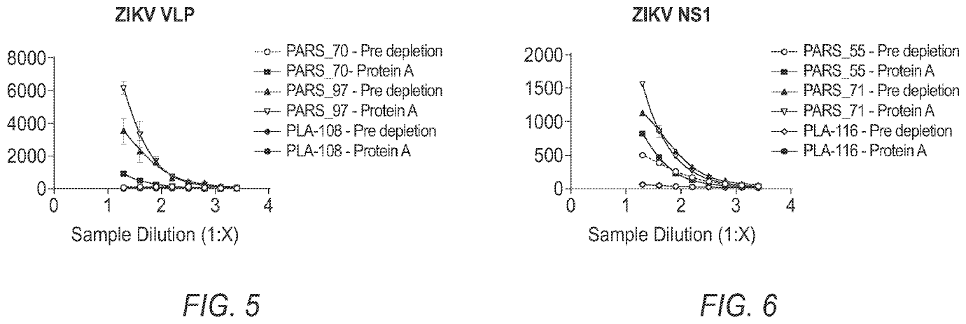

FIG. 5 shows the application of the present method to IgG3 antibodies specific for Zika Virus VLP. It could be demonstrated that the method according to the invention also resulted in an increased sensitivity for such antibodies.

FIG. 6 shows that the present method could be successfully applied to IgG3 antibodies specific for Zika NS1 protein. The method according to the invention resulted in increased sensitivity compared to a method lacking the prior depletion of antibodies other than IgG3 from the sample.

1. A method for determining the presence and/or amount of a human IgG3 antibody specific for a flavivirus antigen in a sample comprising the steps of:
Step 1: contacting an amount of the sample with Protein A coupled to beads to allow binding of human IgG1, human IgG2, human IgG4, human IgA, human IgE and human IgM antibodies to the Protein A coupled to the beads;
Step 2: separating the human IgG1, human IgG2, human IgG4, human IgA, human IgE and human IgM antibodies bound to the Protein A coupled to the beads from the remaining sample and thereby producing a human IgG3 antibody enriched supernatant;
Step 3: removing the human IgG3 antibody enriched supernatant from the human IgG1, human IgG2, human IgG4, human IgA, human IgE and human IgM antibodies bound to the Protein A coupled to the beads;
Step 4: contacting a virus antigen with the human IgG3 antibody enriched supernatant to allow binding of the human IgG3 antibody to the flavivirus antigen;
Step 5: contacting the human IgG3 antibody bound to the virus antigen with a primary IgG specific antibody or a primary IgG3 specific antibody to allow binding of the primary IgG specific antibody or the primary IgG3 specific antibody to the human IgG3 antibody bound to the flavivirus antigen;
Step 6: contacting the bound primary IgG3 specific antibody or the bound primary IgG specific antibody of step 4 with a labelled secondary antibody being specific for the primary IgG3 specific antibody or the primary IgG specific antibody to allow binding of the labelled secondary antibody to the primary antibody; and
Step 7: detecting a signal from the labelled secondary antibody bound to the primary antibody in step 5, wherein the signal is indicative for the presence and/or amount of the labelled secondary antibody and wherein the presence and/or amount of the labelled secondary antibody is indicative for the presence and/or amount of human IgG3 antibody specific for a flavivirus antigen in the sample;
or wherein Steps 5 to 7 are replaced by:
Step 5′: contacting the human IgG3 antibody bound to the virus antigen with a labelled primary IgG specific antibody or a labelled primary IgG3 specific antibody to allow binding of the labelled primary IgG specific antibody or the labelled primary IgG3 specific antibody to the human IgG3 antibody bound to the flavivirus antigen; and
Step 6′: detecting a signal from the labelled primary IgG specific antibody or a labelled primary IgG3 specific antibody bound to the flavivirus antigen in step 5′, wherein the signal is indicative for the presence and/or amount of the labelled primary IgG specific antibody or a labelled primary IgG3 specific antibody and wherein the presence and/or amount of the labelled primary antibody is indicative for the presence and/or amount of human IgG3 antibody specific for a flavivirus antigen in the sample.

2. The method of claim 1, wherein the IgG3 antibody is specific for a dengue virus antigen or a Zika virus antigen.

3. The method according to claim 1, wherein the antigen is selected from the group consisting of virus like particle (VLP), non-structural protein 1, envelope protein, pre-membrane protein, membrane protein, capsid protein, non-structural protein 2A, non-structural protein 2B, non-structural protein 3, non-structural protein 4A, non-structural protein 4B, and non-structural protein 5 and any derivative thereof, preferably the antigen is VLP.

4. The method according to claim 1, wherein step 1 is carried out in a plate or tube format, preferably in 96 well microplates.

5. The method according to claim 1, wherein the protein A is coupled to magnetic beads and the separating step 2 is performed by applying magnetic force.

6. The method according to claim 1, wherein the protein A is coupled to beads and the separating step 2 is performed by gravitational force, preferably by applying a step of centrifugation.

7. The method according to claim 1, wherein the human IgG3 antibody is enriched in the IgG3 supernatant compared to the content in the starting sample by a factor of at least 2, preferably at least 5, more preferably at least 10.

8. The method according to claim 1, wherein the label of the secondary antibody in step 6 includes a chemiluminescent, fluorescent or an enzyme label.

9. The method according to claim 1, wherein the label of the primary antibody in step 5′ includes a chemiluminescent, fluorescent or an enzyme label.

10. The method according to claim 8, wherein the enzyme is horseradish peroxidase, alkaline oxidase or glucose oxidase.

11. The method according to claim 10, wherein the label is a fluorescence label selected from the group consisting of xanthene, fluorescein isothiocyanate, rhodamine, phycoerythrin, cyanine, coumarin, and any derivative thereof.

12. The method according to claim 1, wherein steps 4 to 6 are performed in the format of a sandwich assay.

13. The method according to claim 1, wherein steps 4 to 7 are performed in microplate format, preferably in the format of 96 well plates.

14. The method according to claim 1, wherein steps 4, 5′ and 6′ are performed in microplate format, preferably in the format of 96 well plates.

15. A method for increasing the sensitivity of a method for determining the presence and/or amount of the human IgG3 antibody specific for a flavivirus antigen in a sample comprising performing a step of pre-treatment of the sample containing human IgG3 specific for a flavivirus antigen and one or more of human IgG1, human IgG2, human IgG4, human IgA and human IgM antibodies with immobilized or immobilizable Protein A.

16. The method according to claim 15, wherein the Protein A is coupled to beads.

17. The method according to claim 15, wherein the sample is a blood sample from a human subject, more preferably the sample is from human serum.

18. A method for determining the human IgG3 response to a flaviviral vaccine, preferably a dengue or Zika vaccine comprising performing the method according to any one of claim 1.

19. The method according to claim 18, wherein the human IgG3 response to at least one dengue serotypes selected from DENV1, DENV2, DENV3 and DENV4, preferably to each of the different dengue serotypes is determined.

20. A method for the in vitro diagnosis of a flavivirus infection in a human subject within the last six months, preferably a dengue virus or a Zika virus infection, comprising performing the method according to claim 1.

21. The method according to claim 20, wherein the flavivirus infection is an infection by at least one dengue serotype selected from DENV1, DENV2, DENV3 and DENV4, or a Zika virus infection.

22. The method according to claim 16, wherein the human subject is infected by at least two different flaviviruses, preferably dengue virus and Zika virus.
